(19) United States
(12) Patent Application Publication
(10) Pub. No.: US 2024/0050188 A1
(43) Pub. Date: Feb. 15, 2024

(54) ELECTRIC ILLUMINATION ARM MECHANISM BASED ON SLIT LAMP MICROSCOPE

(71) Applicant: CHONGQING KANGHUA RUIMING SCIENCE TECHNOLOGY CO., LTD, ChongQing (IN)

(72) Inventor: Wang YI, Chongqing (CN)

(73) Assignee: CHONGQING KANGHUA RUIMING SCIENCE TECHNOLOGY CO., LTD, ChongQing (CN)

(21) Appl. No.: 18/366,720

(22) Filed: Aug. 8, 2023

(30) Foreign Application Priority Data
Aug. 9, 2022 (CN) 202211040438.3

(51) Int. Cl. A61B 90/35; A61B 3/135; A61B 3/00

(52) U.S. Cl. CPC A61B 90/35; A61B 3/135; A61B 3/0008

(57) ABSTRACT

An electric illumination arm mechanism based on a slit lamp microscope is characterized in that it comprises a lifting arm, an illumination arm and an electric drive mechanism; The electric drive mechanism is fixed on the connecting part of the lifting arm and is used to drive the illumination arm to swing to realize the electric swing of the illumination arm. The invention has the following beneficial effects: first, realize the electric illumination arm function of the slit lamp microscope; Second, electric control can improve the accuracy of the swing angle of the illumination arm, which is conducive to improving the detection efficiency. Third, it is compatible with the manual illumination arm function. Fourth, it is convenient to realize intelligent operation and remote operation of the whole slit lamp microscope.

### TECHNICAL FIELD

The invention relates to the field of medical equipment, in particular to an electric illumination arm mechanism based on slit lamp microscope.

### BACKGROUND TECHNOLOGY

The eye is the most important organ in human senses. About 80% of the knowledge in the brain is acquired through the eye. Eyes are used to read, read, appreciate pictures, see people, and appreciate beautiful scenery.

Slit lamp microscope is an indispensable important instrument for ophthalmic examination, and is also the most widely used basic instrument for ophthalmic routine examination. The state requires that slit lamp be mandatory in ophthalmic diagnosis and treatment, ophthalmic health care, optometry and other industries.

In all the world, from the smallest facial features clinic to the largest ophthalmic hospital to the top ophthalmic disease research institutions, the ophthalmic slit lamp microscope is the most professional equipment for examining the eyelid, conjunctiva, sclera, cornea, anterior chamber, iris, pupil, lens and the anterior ⅓ vitreous body, and determining the location, nature, size and depth of the lesion.

Ophthalmic slit lamp microscopes are widely used by medical institutions, ophthalmic health care institutions and eyewear chain stores. The expansion and improvement of the functions of the instruments play an important role in human eye health.

The structure of slit lamp is mainly composed of four parts: slit illumination mechanism, microscope optical body, mobile platform mechanism, and chin rest bracket.

When using slit lamp to check some items, it is necessary to swing the lighting arm to a certain angle. For example, the eyelids, eyelids and eyelashes of the inspection items need to swing the lighting arm to 45 degrees left and right respectively, and the lens needs to swing the lighting arm to 10 to 45 degrees left and right respectively. The existing stage slit lamp swing lighting arm adopts the method of manually swing the lighting arm, which angle is required, and manually swing the lighting arm to that angle. Its advantages are simple structure, direct and effective. The disadvantage is that the accuracy is poor, which requires the operator to have certain skill level in equipment operation, which increases the burden of the operator. For example, when the operator manually swings the lighting arm to an angle, the angle will be fine-tuned in order to find the best angle. The manual operation will cause repeated fine-tuning, which will increase the detection time. It may also be that the position is not the best angle.

The patent is to solve the problem of electric swing and swing accuracy of the lighting arm of the slit lamp microscope, and is compatible with the manual swing of the illumination arm. At the same time, it solves a problem for realizing the comprehensive electric and intelligent slit lamp.

### SUMMARY OF THE INVENTION

In view of the shortcomings of the existing technology, the invention proposes an electric illumination arm mechanism based on a slit lamp microscope. The specific technical scheme is as follows:

An electric illumination arm mechanism based on slit lamp microscope, which is characterized in that it comprises a lifting arm, an illumination arm and an electric drive mechanism;

The electric drive mechanism is fixed on the connecting part of the lifting arm, which is used to drive the illumination arm to swing and realize the electric swing of the illumination arm.

Further, the electric illumination arm mechanism can also realize the swing of the illumination arm manually.

Further, the electric drive mechanism is a motor-driven combined and separated transmission device, which drives the lighting arm at the connecting end of the combined and separated transmission device to swing.

Further, the electric drive mechanism comprises a bracket, a motor, a combined and separated transmission device, a driving gear and a driven gear. The motor is installed on the bracket, the combined and separated transmission device is installed on the output end of the motor, the driving gear is installed on the combined and separated transmission device, and the driven gear is installed on the illumination arm.

Further, the combination and separation transmission device controls the combination and separation of the transmission part through power-on and power-off.

When the combined and separated transmission device is powered on, the transmission part is combined, and the motor drives the lighting arm to swing; When the combined and separated transmission device is powered off, the transmission part is separated, and the illumination arm swings manually.

The combination and separation transmission device can also be used to manually control the combination and separation of transmission parts.

Further, the electric drive mechanism can be a gear drive mechanism, a chain drive mechanism, a belt drive mechanism, a cam drive mechanism, a connecting rod drive mechanism or a worm gear drive mechanism.

The beneficial effects of the invention are as follows: First, realize the electric illumination arm function of the slit lamp microscope;

Second, electric control can improve the accuracy of the swing angle of the illumination arm, which is conducive to improving the detection efficiency.

Third, it is compatible with manual lighting arm function.

Fourth, it is convenient to realize intelligent operation and remote operation of the whole slit lamp microscope.

### DESCRIPTION OF ATTACHED DRAWINGS

FIG. 1 is the main sectional view of the patent structure;

FIG. 2 is a partial enlarged view of A in FIG. 1

FIG. 3 is the sectional view of B-B structure of the patent;

FIG. 4 is an exploded view of this patent

FIG. 5 shows the structure of the patent;

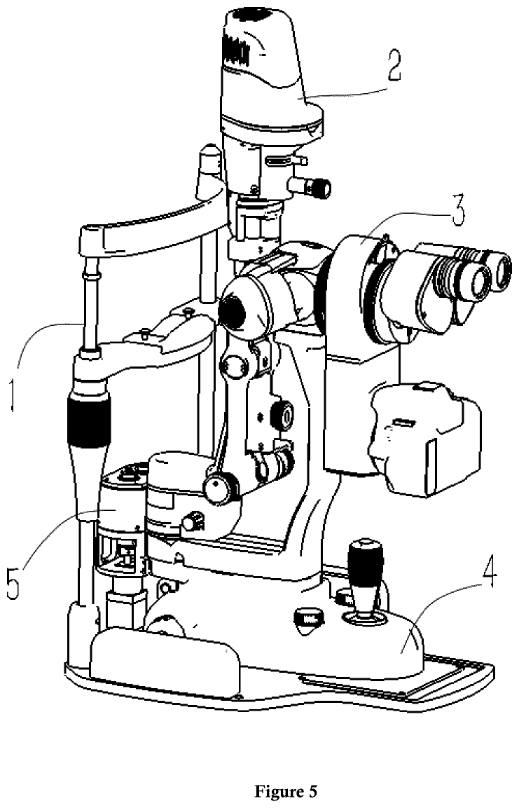

FIG. 6 is the logic block diagram of this patent;

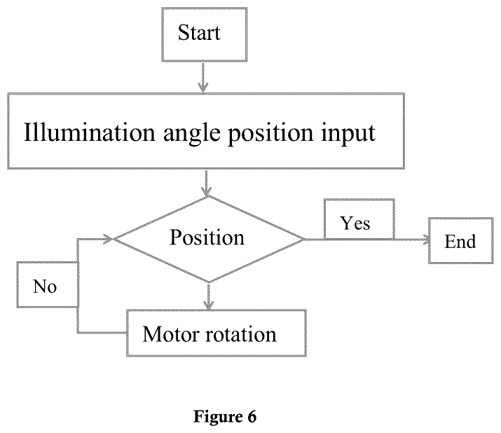

The attached drawings in the figure are marked as follows: chin rest bracket of slit lamp 1, slit lamp illumination mechanism 2, microscope optical body 3, slit lamp platform moving mechanism 4, electric illumination arm mechanism 5, lifting arm 201, lifting arm mandrel 202, rotating shaft 203, motor 204, bracket 205, electromagnetic clutch 206, release plate 306A, driving gear 207, rotating shaft 208, bearings 209, 210, 211, coupling 212, locking rings 213, 214, 219, illumination arm 215, driven gear 216, Driven gear 217, absolute value encoder 218.

### Specific Implementation Mode

The following is a detailed description of the preferred embodiments of the invention in combination with the attached drawings, so that the advantages and features of the invention can be more easily understood by those skilled in the art, and thus the scope of protection of the invention can be more clearly defined.

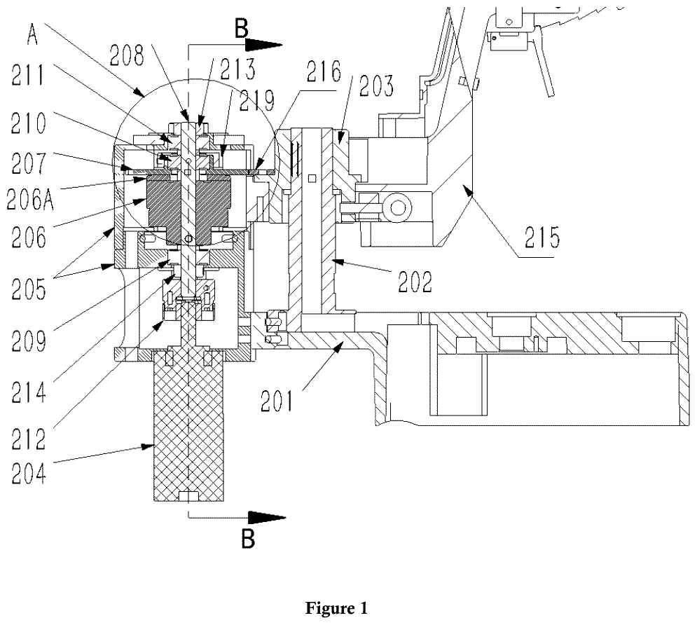

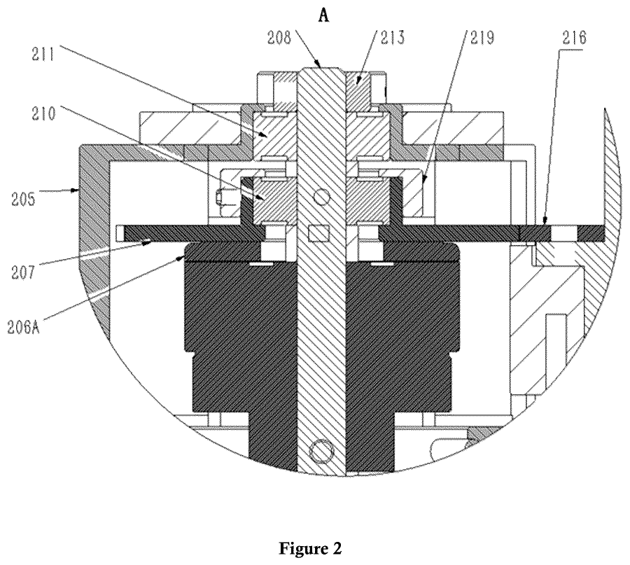

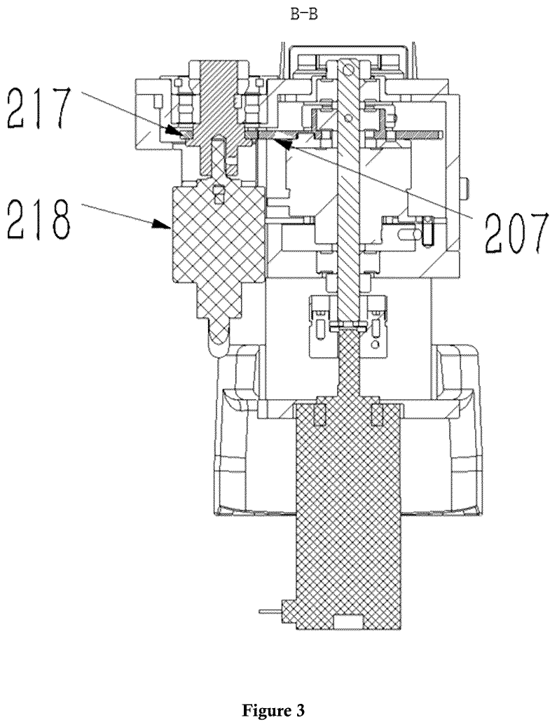

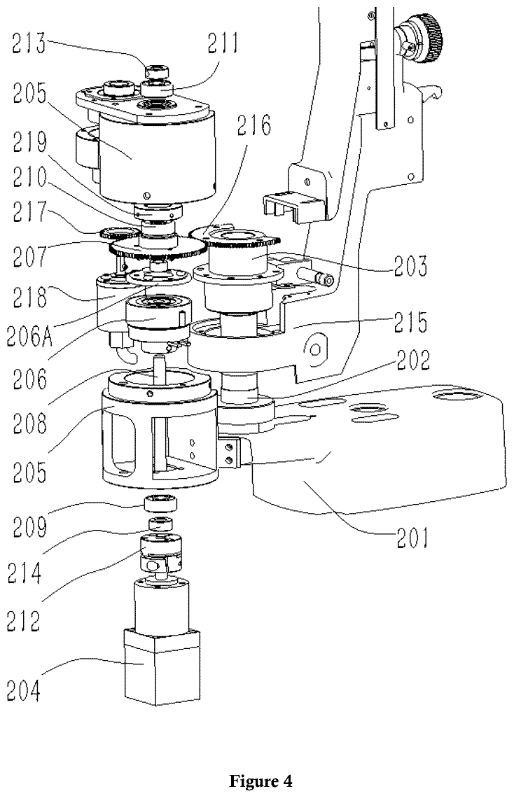

As shown in FIGS. 1 to 4: an automatic and manual illumination arm mechanism based on slit lamp microscope, including lifting arm 201, bracket 205, illumination arm 215, rotating shaft 203, lifting arm mandrel 202.

The lifting arm mandrel 202 is connected above the lifting arm 201, the driven gear 211 is installed on the rotating shaft 203, the rotating shaft 203 is connected with the illumination arm 215, the rotating shaft 203 is placed on the lifting arm mandrel 202, and the illumination arm rotates freely around the mandrel 202.

A bracket 205 is connected on the side of the lifting arm 201, and a motor 204 is arranged in the bracket 205. The output end of the motor 204 is connected to the shaft 208 through the coupling 212. The shaft 208 passes through the inner ring of the bearing 209 first, and then through the inner hole of the electromagnetic clutch 206. The outer ring of the bearing 209 is fixed on the bracket 205, and the inner ring of the bearing 209 is fixed on the shaft 208 through the locking ring 214, and the electromagnetic clutch 206 is fixed on the shaft 208 with its own locking ring, The clutch plate 206A of the electromagnetic clutch 206 is connected with the driving gear 207, and the electromagnetic clutch 206 has a clearance of 0.1˜0.15 mm with the clutch plate 206A, Fix the outer ring of bearing 210 on the driving gear 207 through the locking ring 219, the shaft 208 passes through the inner hole of bearing 210 again, the inner ring of bearing 210 is fixed on the shaft 208, the shaft 208 finally passes through the inner hole of bearing 211, the outer ring of bearing 211 is fixed on the bracket 205, and the inner ring of bearing 211 is fixed on the shaft 208 through the locking ring 213, then the shaft 208 and the electromagnetic clutch 206 both rotate synchronously with the motor 204, and magnetic force is generated when the coil of electromagnetic clutch 206 is energized, The magnetic force acts on the clutch plate 206A. At this time, the electromagnetic clutch 206 and the clutch plate 206A are engaged, The clutch plate 206A rotates with the electromagnetic clutch 206 under the magnetic force. The driving gear 207 connected to the clutch plate 206A rotates together to drive the driven gear 216 to rotate. The lighting arm 215 rotates with the driven gear 216 to realize the electric function of the lighting arm 215. When the coil of the electromagnetic clutch 206 is powered off, the electromagnetic clutch 206 and the clutch plate 206A are in the separation state, the electromagnetic clutch 206 idles, the driving gear 207, the driven gear 216, and the lighting arm 215 rotate freely, and the original manual lighting arm function is retained. The driving gear 207 keeps engaged with the driven gear 217. The driven gear 217 is connected with the encoder 218. The encoder 218 is a device that compiles and converts signals (such as bit streams) or data into signal forms that can be used for communication, transmission and storage. The encoder converts angular displacement or linear displacement into electrical signals. The former is called code disk, and the latter is called code ruler. According to the readout mode, the encoder can be divided into contact type and non-contact type; According to the working principle, encoders can be divided into two types: incremental type and absolute type. The incremental encoder converts the displacement into a periodic electrical signal, and then converts this electrical signal into a counting pulse. The number of pulses represents the displacement. Each position of the absolute encoder corresponds to a certain digital code, so its indication is only related to the starting and ending positions of the measurement, but not to the intermediate process of the measurement. This implementation case uses an absolute value encoder 218, which corresponds to a circle. Each reference angle sends a unique binary value corresponding to the angle, and multiple positions can be recorded and measured through the external circle recording device. Encoder 218 can detect the position of main driving gear 207 and driven gear 217 in real time, thus detecting the position of lighting arm 215.

The driving gear 207 remains engaged with the driven gear 211.

The working principle of the invention: the output torque of the motor is transmitted to the rotating shaft to drive the electromagnetic clutch on the rotating shaft to rotate. When the electromagnetic clutch is energized, the clutch plate and the driving gear rotate with the electromagnetic clutch under the magnetic force, and the driving gear drives the driven gear to rotate, thus driving the illumination arm on the driven gear to swing and realizing the electric swing of the illumination arm; The driving gear drives another driven gear on the encoder to rotate. Through the encoder position detection, the swing angle of the lighting arm can be detected in real time, and the swing angle of the illumination arm can be controlled.

When it is necessary to manually swing the illumination arm, the coil of the electromagnetic clutch is powered off, the electromagnetic clutch and the clutch plate are separated, the driving gear and the driven gear rotate freely, and the required illumination arm angle can be reached by manually swinging the illumination arm,

When the electric swing illumination arm is required, the coil of the electromagnetic clutch is energized to generate magnetic force. Under the effect of magnetic force, the electromagnetic clutch and the clutch plate are in the engagement state. Through the motor drive and encoder control, the required illumination arm angle is reached.

For those skilled in the art, it is obvious that the invention is not limited to the details of the above exemplary embodiments, and can be realized in other specific forms without departing from the spirit or basic features of the invention. Therefore, from any point of view, the embodiments should be regarded as exemplary and non-restrictive. The scope of the invention is limited by the appended claims rather than the above description. Therefore, it is intended to include all changes falling within the meaning and scope of the equivalent elements of the claims in the invention. Any reference mark in the claims shall not be regarded as limiting the claims involved.

In addition, it should be understood that although this specification is described in accordance with the embodiments, not each embodiment only contains an independent technical solution. This description of the specification is only for the sake of clarity. Those skilled in the art should take the specification as a whole, and the technical solutions in each embodiment can also be properly combined to form other embodiments that can be understood by those skilled in the art.

We claim:

1. An electric illumination arm mechanism based on slit lamp microscope, which is characterized in that the electric illumination arm comprises a lifting arm, an illumination arm and an electric drive mechanism; and
the electric drive mechanism is fixed on the connecting part of the lifting arm, which is used to drive the illumination arm to swing and realize the electric swing of the illumination arm.

2. The electric illumination arm mechanism based on slit lamp microscope according to claim 1, wherein the electric illumination arm mechanism realizes the manual swing of the illumination arm.

3. The electric illumination arm mechanism based on slit lamp microscope according to claim 1, wherein the electric driving mechanism is a motor-driven combined and separated transmission device, which drives the illumination arm at the connecting end of the combined and separated transmission device to swing.

4. The electric illumination arm mechanism based on slit lamp microscope according to claim 1, wherein the electric drive mechanism comprises a bracket, a motor, a combined and separated transmission device, a driving gear and a driven gear, the motor is installed on the bracket, the combined and separated transmission device are installed on the output end of the motor, and the driving gear is installed on the combined and separated transmission device and the driven gear is installed on the illumination arm.

5. The electric illumination arm mechanism based on slit lamp microscope according to claim 3, wherein the combination and separation transmission device controls the combination and separation of the transmission part through power-on and power-of and when the combined and separated transmission device is powered on, the transmission part is combined, and the motor drives the illumination arm to swing and when the combined and separated transmission device is powered off, the transmission part is separated, and the illumination arm swings manually and combination and separation transmission device is used to manually control the combination and separation of transmission parts.

6. The electric illumination arm mechanism based on slit lamp microscope according to claim 1, wherein the electric driving mechanism is a gear drive mechanism, a chain drive mechanism, a belt drive mechanism, a cam drive mechanism, a connecting rod drive mechanism or a worm gear and worm drive mechanism.
